(19) United States
(12) Patent Application Publication
(10) Pub. No.: US 2021/0386689 A1
(43) Pub. Date: Dec. 16, 2021

(54) METHODS OF TREATING CENTRAL NERVOUS SYSTEM DISORDERS

(71) Applicant: Sunovion Pharmaceuticals Inc., Marlborough, MA (US)

(72) Inventor: Jing LIN, Marlborough, MA (US)

(73) Assignee: Sunovion Pharmaceuticals Inc., Marlborough, MA (US)

(21) Appl. No.: 17/286,155

(22) PCT Filed: Oct. 30, 2019
(86) PCT No.: PCT/US2019/058756

§ 371 Date: Apr. 16, 2021

Related U.S. Application Data
(60) Provisional application No. 62/753,127, filed on Oct. 31, 2018.

(51) Int. Cl. A61K 31/135; A61K 31/137; A61K 31/536; A61K 31/55; A61K 31/496; A61K 31/427

(52) U.S. Cl. CPC A61K 31/135; A61K 31/137; A61K 31/427; A61K 31/55; A61K 31/496; A61K 31/536

(57) ABSTRACT

Provided herein are methods of treating central nervous system (CNS) disorders (e.g., Binge Eating Disorder (BED), Attention-Deficit/Hyperactivity Disorder (ADHD), etc.) with a dasotraline therapy while avoiding adverse drug interactions.

### CROSS REFERENCE TO RELATED APPLICATIONS

This application claims priority to U.S. Provisional Patent Application No. 62/753,127, filed Oct. 31, 2018, the content of which is hereby incorporated by reference herein in its entirety.

### FIELD

Provided herein are methods of treating central nervous system (CNS) disorders (e.g., Binge Eating Disorder (BED), Attention-Deficit/Hyperactivity Disorder (ADHD), etc.) with a dasotraline therapy while avoiding adverse drug interactions.

### BACKGROUND

The United States Food and Drug Administration (FDA) recognizes that patients frequently use more than one medication at a time, which poses a potential risk for adverse drug interactions (e.g., drug-drug interactions). (FDA's Clinical Drug Interaction Studies—Study Design, Data Analysis, and Clinical Implications Guidance for Industry; October 2017.) Many factors are involved in determining whether a particular medicament is at risk for a potential drug-drug interaction. One of the many factors to consider is the medicament's metabolism.

Two substances (e.g., medicament, food, etc.) that are metabolized by/substrates of the same enzyme may have an effect on each other's metabolism. For example, some statins are metabolized by the same enzyme that is inhibited by compounds that naturally exist in grapefruit juice, which poses a potential risk for patients receiving a statin therapy while also drinking grapefruit juice. (Lee, Jonathan W., et al. Grapefruit Juice and Statins, The American Journal of Medicine 129(1): 26-29 (2016).) In particular, the statins atorvastatin, simvastatin, and lovastatin are metabolized by the enzyme Cytochrome P450 (CYP) 3A4 (CYP3A4), but CYP3A4 is inhibited by grapefruit juice's furanocoumarin compounds bergamottin and 6′,7′-dihydroxybergamottin (DHB). Thus, if a patient consumes one of these statins and grapefruit juice at the same time, the patient will receive a higher exposure to the statin because the grapefruit juice is inhibiting/preventing the CYP3A4 enzyme from metabolizing the statin. However, a patient consuming a different statin, such as fluvastatin or rosuvastatin, which are primarily metabolized by CYP2C9, would not experience the same increase of statin exposure if consumed concomitantly with grapefruit juice.

Therefore, ascertaining a medicament's potential risk for drug-drug interactions and devising methods of reducing the potential risk is desirable to ensure that a medicament is safe and effective for patients.

Dasotraline is a compound undergoing clinical studies to research treatments for Binge Eating Disorder (BED) and Attention-Deficit/Hyperactivity Disorder (ADHD).

### SUMMARY

Accordingly, provided herein are methods of treating central nervous system (CNS) disorders (e.g., Binge Eating Disorder (BED), Attention-Deficit/Hyperactivity Disorder (ADHD), etc.) with a dasotraline therapy while avoiding adverse drug interactions.

Provided herein are methods of treating a central nervous system (CNS) disorder, in a subject in need thereof, comprising concomitantly administering dasotraline, or a pharmaceutically acceptable salt thereof, and a CYP2B6 substrate.

In some embodiments, provided is a method of treating a central nervous system (CNS) disorder with a therapeutically effective amount of dasotraline, or a pharmaceutically acceptable salt thereof, in a subject in need thereof and also in need of a therapeutically effective amount of a CYP2B6 substrate, comprising decreasing the dose or dose frequency of the CYP2B6 substrate during concomitant administration with dasotraline, or a pharmaceutically acceptable salt thereof.

In some embodiments, provided is a method of treating a central nervous system (CNS) disorder, in a subject in need thereof, comprising administering a therapeutically effective amount of dasotraline, or a pharmaceutically acceptable salt thereof, and avoiding a CYP2B6 substrate.

In some embodiments, provided is a method of treating a central nervous system (CNS) disorder with a therapeutically effective amount of dasotraline, or a pharmaceutically acceptable salt thereof, in a subject in need thereof and also in need of a CYP2B6 substrate, comprising avoiding the CYP2B6 substrate during administration of dasotraline, or a pharmaceutically acceptable salt thereof.

In some embodiments, provided is a method of treating a central nervous system (CNS) disorder, in a subject in need thereof, comprising administering a therapeutically effective amount of dasotraline, or a pharmaceutically acceptable salt thereof, and discontinuing administration of a CYP2B6 substrate.

In some embodiments, provided is a method of treating a central nervous system (CNS) disorder with a therapeutically effective amount of dasotraline, or a pharmaceutically acceptable salt thereof, in a subject in need thereof and also in need of a CYP2B6 substrate, comprising discontinuing administration of the CYP2B6 substrate during administration of dasotraline, or a pharmaceutically acceptable salt thereof.

In some embodiments, provided is a method of treating a central nervous system (CNS) disorder while reducing potential drug-drug interactions, in a subject in need thereof, comprising concomitantly administering a therapeutically effective amount of dasotraline, or a pharmaceutically acceptable salt thereof, and a decrease from a normally effective amount of a CYP2B6 substrate.

In some embodiments, provided is a method of administering a therapeutically effective amount of dasotraline, or a pharmaceutically acceptable salt thereof, to a subject in need thereof, and also in need of a CYP2B6 substrate, comprising concomitantly administering the therapeutically effective amount of dasotraline, or a pharmaceutically acceptable salt thereof, and at least 10% less of the CYP2B6 substrate.

In some embodiments, provided is a method of treating a central nervous system (CNS) disorder, in a subject in need thereof, comprising concomitantly administering an increase from a therapeutically effective dose of dasotraline, or a pharmaceutically acceptable salt thereof, and a therapeutically effective amount of a CYP2B6 inducer.

In some embodiments, provided is a method of treating a central nervous system (CNS) disorder with a therapeutically effective dose of dasotraline, or a pharmaceutically acceptable salt thereof, in a subject in need thereof and also in need of a therapeutically effective amount of a CYP2B6 inducer, comprising increasing the dose or dose frequency of dasotraline during concomitant administration with the CYP2B6 inducer.

In some embodiments, provided is a method of treating a central nervous system (CNS) disorder, in a subject in need thereof, comprising administering once daily 2 mg-8 mg of dasotraline, or a pharmaceutically acceptable salt thereof, and avoiding a CYP2B6 inducer.

In some embodiments, provided is a method of treating a central nervous system (CNS) disorder with 2 mg-8 mg of dasotraline, or a pharmaceutically acceptable salt thereof, once daily in a subject in need thereof and also in need of a CYP2B6 inducer, comprising avoiding the CYP2B6 inducer during administration of dasotraline, or a pharmaceutically acceptable salt thereof.

In some embodiments, provided is a method of treating a central nervous system (CNS) disorder, in a subject in need thereof, comprising administering once daily 2 mg-8 mg of dasotraline, or a pharmaceutically acceptable salt thereof, and discontinuing administration of a CYP2B6 inducer.

In some embodiments, provided is a method of treating a central nervous system (CNS) disorder with 2 mg-8 mg of dasotraline, or a pharmaceutically acceptable salt thereof, once daily in a subject in need thereof and also in need of a CYP2B6 inducer, comprising discontinuing administration of the CYP2B6 inducer during administration of dasotraline, or a pharmaceutically acceptable salt thereof.

In some embodiments, provided is a method of treating a central nervous system (CNS) disorder while reducing potential drug-drug interactions, in a subject in need thereof, comprising concomitantly administering an increase of dasotraline, or a pharmaceutically acceptable salt thereof, and an effective amount of a CYP2B6 inducer.

In some embodiments, provided is a method of administering dasotraline, or a pharmaceutically acceptable salt thereof, to a subject in need thereof, and also in need of a CYP2B6 inducer, comprising concomitantly administering the CYP2B6 inducer and more than a normally effective amount of dasotraline, or a pharmaceutically acceptable salt thereof.

In some embodiments, provided is a method of treating a central nervous system (CNS) disorder, in a subject in need thereof, comprising concomitantly administering once daily 2 mg-8 mg of dasotraline, or a pharmaceutically acceptable salt thereof, and a therapeutically effective amount of a CYP2B6 inhibitor, wherein the CYP2B6 inhibitor is a medicament.

### DETAILED DESCRIPTION

The description herein sets forth details to provide an understanding of various embodiments of the invention, and is made with the understanding that the provided disclosures are an exemplification of the claimed subject matter without intending to limit the claims to specific embodiments. Accordingly, specific embodiments disclosed herein may be combined with other specific embodiments disclosed herein, including specific embodiments under various headings, which are provided for convenience and organization, but are not to be construed to limit the claims in any way.

All published documents cited herein are hereby incorporated by reference in their entirety.

### Definitions

Unless defined otherwise, all technical and scientific terms used herein have the same meaning as commonly understood by one of ordinary skill in the art.

As used herein, the singular forms “a”, “an” and “the” are intended to include the plural forms as well, unless the context clearly indicates otherwise.

As used herein, the terms “comprising” and “including” or grammatical variants thereof are to be taken as specifying the stated features, integers, steps or components but do not preclude the addition of one or more additional features, integers, steps, components or groups thereof. These terms encompass the term “consisting of”.

As used herein, a “drug-drug interaction” or grammatical variants thereof is a modification of the effect of a drug when administered with another drug. The effect may be an increase or a decrease in the action of either substance, or it may be an adverse effect that is not normally associated with either drug. The particular interaction may be the result of a chemical-physical incompatibility of the two drugs, a change in the rate of metabolism of either drug, or a change in the rate of absorption or the quantity absorbed in the body, the binding ability of either drug, or an alteration in the ability of receptor sites and cell membranes to bind either drug. In some embodiments of a drug-drug interaction, a change in exposure of a medicament is observed, wherein the change is primarily caused by concomitant administration of a separate medicament.

As used herein, “Active Pharmaceutical Ingredient” or “API” refers to a therapeutically active ingredient of a medicament.

As used herein, “at least XX %” refers to XX % or more. For example, “at least 10%” includes 10%, 11%, 12%, etc.

As used herein, “avoid” (or grammatical variants thereof) refers to “refraining from.” For example, to avoid administering a CYP2B6 substrate, includes refraining from administering a CYP2B6 substrate. In some embodiments, to avoid administration includes the periods of time from discontinuing administration of a medicament to complete elimination of the discontinued medicament by a subject. In some embodiments, at least two medicaments can be concomitantly administered to a subject in need thereof, wherein one medicament is avoided, but has not been completely eliminated by the subject.

As used herein, “central nervous disorder” or “CNS” or specifically identified disorders disclosed herein, (e.g. CNS disorders) refer to the disorder as defined in the Diagnostic and Statistical Manual of Mental Disorders, Fifth Edition (DSM-5).

As used herein, “concomitant administration” refers to administration of two separate medicaments within a period of time that is insufficient for partial or complete elimination of at least one of the medicaments. In some embodiments, concomitant administration encompasses administration of a second medicament within the t½ of a first-administered drug. For example, concomitant administration of dasotraline and a CYP2B6 interactor includes administering dasotraline and the CYP2B6 interactor during the periods of time before either dasotraline elimination and/or the CYP2B6 interactor elimination from a subject.

As used herein, “CYP2B6” refers to the human enzyme cytochrome P450, family 2, subfamily B, member 6.

As used herein, “CYP2B6 Interactor” refers to a CYP2B6 substrate, CYP2B6 inducer, and CYP2B6 inhibitor.

As used herein, “CYP2B6 substrate” refers to a medicament that is, at least partially, metabolized by CYP2B6.

As used herein, “CYP2B6 inducer” refers to a medicament that, at least partially, increases the metabolic activity of CYP2B6.

As used herein, “CYP2B6 inhibitor” refers to a medicament that, at least partially, decreases the metabolic activity of CYP2B6. Inhibition of CYP enzymes (e.g., CYP2B6) can be classified into two categories: reversible/direct or time dependent/metabolism-dependent/indirect. Reversible (direct) inhibition involves rapid association and dissociation of a medicament and CYP enzymes (e.g., CYP2B6). Typically, time-dependent inhibition (TDI) results from irreversible covalent binding or quasi-irreversible non-covalent tight binding of a chemically reactive intermediate to the enzyme that catalyzes its formation, resulting a loss of enzyme function.

As used herein, “dasotraline therapy” refers to a treatment regimen comprising administration of dasotraline. Dasotraline therapy may include concomitant administration of other medicaments. In some embodiments, dasotraline therapy refers to a treatment regimen comprising orally administering dasotraline to a subject in need thereof.

As used herein, “elimination” (or grammatical variants thereof) refers to the removal of a medicament from a subject. In some embodiments, elimination refers to the complete removal of a medicament, wherein the plasma concentration of the medicament is not significantly detectable.

As used herein, “exposure” refers to the plasma concentration of a medicament in a subject over time.

As used herein, “normally effective amount” refers to an amount of dasotraline that would be therapeutically effective in the absence of a CYP2B6 inducer or an amount of a CYP2B6 substrate that would be therapeutically effective in the absence of dasotraline.

As used herein, “in need thereof” refers to a subject who would benefit from administration of a medicament. The patient may be suffering from any disease or condition for which the medicament therapy may be useful in ameliorating symptoms. For example, a subject in need of dasotraline includes a subject who would benefit from administration of dasotraline, and may be suffering from any disease or condition for which a dasotraline therapy may be useful in ameliorating symptoms. Such diseases or conditions include central nervous disorders as defined herein.

As used herein, “grammatical variants” refers to the various prefixes and/or suffixes that may be necessary for a word to be properly included in a sentence, but does not significantly change the meaning of the root word. For example, the defined term “administering” includes non-limiting grammatical variants such as “administer,” “administered,” “administration,” and other examples that may be found in the dictionary.

As used herein, “medicament” refers to a substance that can be administered to a subject to treat a disease or condition. A medicament typically includes at least one an active pharmaceutical ingredient and pharmaceutically acceptable excipients. In some embodiments, a medicament comprises at least one active pharmaceutical ingredient. In some embodiments, a medicament comprises at least one active pharmaceutical ingredient and at least one pharmaceutically acceptable excipient.

As used herein, “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. The “subject” may have independently been diagnosed with a disorder as defined herein, may currently be experiencing symptoms associated with disorders or may have experienced symptoms in the past, may be at risk of developing a disorder, or may be reporting one or more of the symptoms of a disorder, even though a diagnosis may not have been made. In some embodiments, the subject is a human who may have independently been diagnosed with a disorder as defined herein, may currently be experiencing symptoms associated with disorders or may have experienced symptoms in the past, may be at risk of developing a disorder, or may be reporting one or more of the symptoms of a disorder, even though a diagnosis may not have been made.

As used herein, “treatment” (or grammatical variants thereof) refer to an approach for obtaining beneficial or desired results including, but not limited to, therapeutic benefit. Therapeutic benefit includes eradication and/or amelioration of the underlying disorder being treated; it also includes the eradication and/or amelioration of one or more of the symptoms associated with the underlying disorder such that an improvement is observed in the subject, notwithstanding that the subject may still be afflicted with the underlying disorder. In some embodiments, “treatment” or “treating” includes one or more of the following: (a) inhibiting the disorder (for example, decreasing one or more symptoms resulting from the disorder, and/or diminishing the extent of the disorder); (b) slowing or arresting the development of one or more symptoms associated with the disorder (for example, stabilizing the disorder and/or delaying the worsening or progression of the disorder); and/or (c) relieving the disorder (for example, causing the regression of clinical symptoms, ameliorating the disorder, delaying the progression of the disorder, and/or increasing quality of life). In some embodiments, treatment can be administered after one or more symptoms have developed. In other embodiments, treatment can be administered in the absence of symptoms. For example, treatment can be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, “therapeutically effective amount” or “effective amount” refers to an amount (e.g., dose) that is effective to elicit the desired biological or medical response, including the amount of a compound that, when administered to a subject for treating a disorder, is sufficient to effect such treatment of the disorder. The effective amount will vary depending on the compound, the disorder, and its severity, and the age, weight, etc. of the subject to be treated. The effective amount may be in one or more doses (for example, a single dose or multiple doses may be required to achieve the desired treatment endpoint). An effective amount may be considered to be given in an effective amount if, in conjunction with one or more other agents, a desirable or beneficial result may be or is achieved. Suitable doses of any co-administered compounds may optionally be lowered due to the combined action, additive or synergistic, of the compound.

Dasotraline

Provided herein are methods of treating central nervous system (CNS) disorders (e.g., Binge Eating Disorder (BED), Attention-Deficit/Hyperactivity Disorder (ADHD), etc.) with a dasotraline therapy while avoiding adverse drug interactions. Dasotraline has the structure:

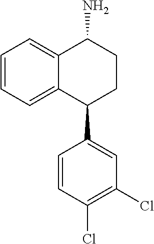 [SMILES: N[C@@H]1CC[C@@H](c2ccc(Cl)c(Cl)c2)c2ccccc21]

and is described, for example, in PCT Publication No. WO2004/024669, the entirety of which is incorporated herein by reference.

Dasotraline is named or identified using other commonly recognized nomenclature systems. For example, dasotraline may be named or identified with common names, systematic names, or non-systematic names. The nomenclature systems that are commonly recognized in the art of chemistry include, but are not limited to, Chemical Abstract Service (CAS) and International Union of Pure and Applied Chemistry (IUPAC). Dasotraline has a CAS Registry Number of 675126-05-3, and an IUPAC name of: (1R,4S)-4-(3,4-dichlorophenyl)-1,2,3,4-tetrahydronaphthalen-1-amine.

Dasotraline may be prepared as a pharmaceutically acceptable salt. Dasotraline may be prepared as a pharmaceutically acceptable HCl salt. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, tosylates, xylenesulfonates, naphthalene-1-sulfonates, naphthalene-2-sulfonates, phenylacetates, phenylpropionates, phenylbutyrates, citrates, lactates, gamma-hydroxybutyrates, glycolates, tartrates, and mandelates. When dosages are given herein, and the dasotraline is administered as a salt, the dose refers to the amount of dasotraline calculated as the free base. Thus 8 mg of dasotraline is equivalent to 9 mg of dasotraline hydrochloride.

Dasotraline has shown efficacy in treating Binge Eating Disorder (BED) [Navia, B., et al. “Dasotraline for the Treatment of Moderate to Severe Binge Eating Disorder in Adults: Results from a Randomized, Double Blind, Placebo-Controlled Study”, Psych Congress, September 16-19 (2017) in New Orleans, La., USA] and Attention-Deficit/Hyperactivity Disorder (ADHD) [Goldman, A., et al. “Dasotraline in Children with Attention Deficit Hyperactivity Disorder: Results of a Randomized, Double-Blind, Placebo-Controlled Study”, 6th World Congress on ADHD, April 20-23 (2017) in Vancouver, British Columbia, Canada]. The entirety of both references is incorporated herein by reference.

Eight clinical studies—including single dose pharmacokinetic (PK), multiple dose PK, efficacy, safety, and tolerability, human abuse liability, absorption, metabolism, and excretion, and safety, efficacy, and tolerability in adults with attention-deficit hyperactivity disorder—were completed with dasotraline. In those studies, single doses ranging from 0.2 mg to 36 mg and multiple doses from 0.5 mg/day to 8 mg/day for 21 to 56 days were evaluated. Dasotraline was slowly absorbed into the systemic circulation, reaching time of maximum observed plasma concentration (tmax) approximately 10 to 12 hours after single administration of doses ranging from 0.2 to 36 mg. The change in maximum observed plasma concentration (Cmax) with dose was nearly dose proportional for the same doses. Dasotraline elimination was slow, with mean apparent terminal elimination half-life (t½) values ranging from 47.1 to 77.0 hours. Dasotraline is extensively metabolized by oxidation and subsequent phase II metabolism. CYP2B6 oxidizes dasotraline to “dasotraline oxime”:

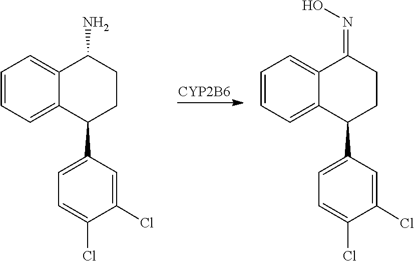

Two substances (e.g., dasotraline and another medicament) that are inhibited or induced by, or substrates of, the same enzyme (e.g., CYP2B6), may have an effect on either substances' pharmacokinetics and/or pharmacodynamics. CYP interactions are recognized as an important cause of drug-drug interactions, some of which have proven fatal (Parkinson and Ogilvie 2008; Ogilvie et al. 2008). Regulatory agencies, such as the FDA, recommend that drug candidates be evaluated in vitro for human CYP enzyme interactions.

Through preclinical and clinical studies, including the Examples disclosed herein, dasotraline was discovered to be extensively metabolized in humans by the enzyme Cytochrome P450 2B6 (CYP2B6). Additionally, dasotraline was discovered to be a time-dependent (irreversible) inhibitor of CYP2B6.

Accordingly, provided herein are methods of treating central nervous system (CNS) disorders (e.g., Binge Eating Disorder (BED), Attention-Deficit/Hyperactivity Disorder (ADHD), etc.) with a dasotraline therapy while avoiding drug-drug interactions.

Methods

Provided herein are methods of treating a central nervous system (CNS) disorder, in a subject in need thereof, comprising concomitantly administering dasotraline, or a pharmaceutically acceptable salt thereof, and a CYP2B6 substrate. In some embodiments, provided is a method of treating a central nervous system (CNS) disorder, in a subject in need thereof, comprising concomitantly administering once daily 2 mg-8 mg of dasotraline, or a pharmaceutically acceptable salt thereof, and an at least 10% decrease from a normally effective amount of a CYP2B6 substrate, wherein the CYP2B6 substrate is bupropion or efavirenz, or a pharmaceutically acceptable salt thereof. In some embodiments, provided is a method of treating a central nervous system (CNS) disorder, in a subject in need thereof, comprising concomitantly administering once daily 2 mg-8 mg of dasotraline, or a pharmaceutically acceptable salt thereof, and an at least 40% decrease from a normally effective amount of a CYP2B6 substrate, wherein the CYP2B6 substrate is bupropion or efavirenz, or a pharmaceutically acceptable salt thereof.

In some embodiments, provided is a method of treating a central nervous system (CNS) disorder with a therapeutically effective amount of dasotraline, or a pharmaceutically acceptable salt thereof, in a subject in need thereof and also in need of a therapeutically effective amount of a CYP2B6 substrate, comprising decreasing the dose or dose frequency of the CYP2B6 substrate during concomitant administration with dasotraline, or a pharmaceutically acceptable salt thereof. In some embodiments, provided is a method of treating a central nervous system (CNS) disorder with 2 mg-8 mg of dasotraline, or a pharmaceutically acceptable salt thereof, once daily in a subject in need thereof and also in need of a therapeutically effective amount of a CYP2B6 substrate, comprising decreasing the dose of the CYP2B6 substrate by at least 10% during concomitant administration with dasotraline, or a pharmaceutically acceptable salt thereof, wherein the CYP2B6 substrate is bupropion or efavirenz, or a pharmaceutically acceptable salt thereof. In some embodiments, provided is a method of treating a central nervous system (CNS) disorder with 2 mg-8 mg of dasotraline, or a pharmaceutically acceptable salt thereof, once daily in a subject in need thereof and also in need of a therapeutically effective amount of a CYP2B6 substrate, comprising decreasing the dose frequency of the CYP2B6 substrate by at least 10% during concomitant administration with dasotraline, or a pharmaceutically acceptable salt thereof, wherein the CYP2B6 substrate is bupropion or efavirenz, or a pharmaceutically acceptable salt thereof. In some embodiments, provided is a method of treating a central nervous system (CNS) disorder with 2 mg-8 mg of dasotraline, or a pharmaceutically acceptable salt thereof, once daily in a subject in need thereof and also in need of a therapeutically effective amount of a CYP2B6 substrate, comprising decreasing the dose of the CYP2B6 substrate by at least 40% during concomitant administration with dasotraline, or a pharmaceutically acceptable salt thereof, wherein the CYP2B6 substrate is bupropion or efavirenz, or a pharmaceutically acceptable salt thereof. In some embodiments, provided is a method of treating a central nervous system (CNS) disorder with 2 mg-8 mg of dasotraline, or a pharmaceutically acceptable salt thereof, once daily in a subject in need thereof and also in need of a therapeutically effective amount of a CYP2B6 substrate, comprising decreasing the dose frequency of the CYP2B6 substrate by at least 40% during concomitant administration with dasotraline, or a pharmaceutically acceptable salt thereof, wherein the CYP2B6 substrate is bupropion or efavirenz, or a pharmaceutically acceptable salt thereof. In some embodiments, provided is a method of treating a central nervous system (CNS) disorder, in a subject in need thereof, comprising administering a therapeutically effective amount of dasotraline, or a pharmaceutically acceptable salt thereof, and avoiding a CYP2B6 substrate. In some embodiments, provided is a method of treating a central nervous system (CNS) disorder, in a subject in need thereof, comprising administering once daily 2 mg-8 mg of dasotraline, or a pharmaceutically acceptable salt thereof, and avoiding a CYP2B6 substrate, wherein the CYP2B6 substrate is bupropion or efavirenz, or a pharmaceutically acceptable salt thereof. In some embodiments, provided is a method of treating a central nervous system (CNS) disorder with a therapeutically effective amount of dasotraline, or a pharmaceutically acceptable salt thereof, in a subject in need thereof and also in need of a CYP2B6 substrate, comprising avoiding the CYP2B6 substrate during administration of dasotraline, or a pharmaceutically acceptable salt thereof. In some embodiments, provided is a method of treating a central nervous system (CNS) disorder with 2 mg-8 mg of dasotraline, or a pharmaceutically acceptable salt thereof, once daily in a subject in need thereof and also in need of a CYP2B6 substrate, comprising avoiding the CYP2B6 substrate during administration of dasotraline, or a pharmaceutically acceptable salt thereof, wherein the CYP2B6 substrate is bupropion or efavirenz, or a pharmaceutically acceptable salt thereof.

In some embodiments, provided is a method of treating a central nervous system (CNS) disorder, in a subject in need thereof, comprising administering a therapeutically effective amount of dasotraline, or a pharmaceutically acceptable salt thereof, and discontinuing administration of a CYP2B6 substrate. In some embodiments, provided is a method of treating a central nervous system (CNS) disorder, in a subject in need thereof, comprising administering once daily 2 mg-8 mg of dasotraline, or a pharmaceutically acceptable salt thereof, and discontinuing administration of a CYP2B6 substrate, wherein the CYP2B6 substrate is bupropion or efavirenz, or a pharmaceutically acceptable salt thereof.

In some embodiments, provided is a method of treating a central nervous system (CNS) disorder with a therapeutically effective amount of dasotraline, or a pharmaceutically acceptable salt thereof, in a subject in need thereof and also in need of a CYP2B6 substrate, comprising discontinuing administration of the CYP2B6 substrate during administration of dasotraline, or a pharmaceutically acceptable salt thereof. In some embodiments, provided is a method of treating a central nervous system (CNS) disorder with 2 mg-8 mg of dasotraline, or a pharmaceutically acceptable salt thereof, once daily in a subject in need thereof and also in need of a CYP2B6 substrate, comprising discontinuing administration of the CYP2B6 substrate during administration of dasotraline, or a pharmaceutically acceptable salt thereof, wherein the CYP2B6 substrate is bupropion or efavirenz, or a pharmaceutically acceptable salt thereof.

In some embodiments, provided is a method of treating a central nervous system (CNS) disorder while reducing potential drug-drug interactions, in a subject in need thereof, comprising concomitantly administering a therapeutically effective amount of dasotraline, or a pharmaceutically acceptable salt thereof, and a decrease from a normally effective amount of a CYP2B6 substrate. In some embodiments, provided is a method of treating a central nervous system (CNS) disorder while reducing potential drug-drug interactions, in a subject in need thereof, comprising concomitantly administering once daily 2 mg-8 mg of dasotraline, or a pharmaceutically acceptable salt thereof, and an at least 10% decrease from a normally effective amount of a CYP2B6 substrate, wherein the CYP2B6 substrate is bupropion or efavirenz, or a pharmaceutically acceptable salt thereof. In some embodiments, provided is a method of treating a central nervous system (CNS) disorder while avoiding a risk of seizure, in a subject in need thereof, comprising concomitantly administering once daily 2 mg-8 mg of dasotraline, or a pharmaceutically acceptable salt thereof, and an at least 10% decrease from a normally effective amount of a CYP2B6 substrate, wherein the CYP2B6 substrate is bupropion or efavirenz, or a pharmaceutically acceptable salt thereof. In some embodiments, provided is a method of treating a central nervous system (CNS) disorder while reducing potential drug-drug interactions, in a subject in need thereof, comprising concomitantly administering once daily 2 mg-8 mg of dasotraline, or a pharmaceutically acceptable salt thereof, and an at least 40% decrease from a normally effective amount of a CYP2B6 substrate, wherein the CYP2B6 substrate is bupropion or efavirenz, or a pharmaceutically acceptable salt thereof. In some embodiments, provided is a method of treating a central nervous system (CNS) disorder while avoiding a risk of seizure, in a subject in need thereof, comprising concomitantly administering once daily 2 mg-8 mg of dasotraline, or a pharmaceutically acceptable salt thereof, and an at least 40% decrease from a normally effective amount of a CYP2B6 substrate, wherein the CYP2B6 substrate is bupropion or efavirenz, or a pharmaceutically acceptable salt thereof.

In some embodiments, provided is a method of administering a therapeutically effective amount of dasotraline, or a pharmaceutically acceptable salt thereof, to a subject in need thereof, and also in need of a CYP2B6 substrate, comprising concomitantly administering the therapeutically effective amount of dasotraline, or a pharmaceutically acceptable salt thereof, and at least 10% less of the CYP2B6 substrate. In some embodiments, provided is a method of administering 2 mg-8 mg of dasotraline, or a pharmaceutically acceptable salt thereof, once daily to a subject in need thereof, and also in need of a CYP2B6 substrate, comprising concomitantly administering 2 mg-8 mg of dasotraline, or a pharmaceutically acceptable salt thereof, once daily and at least 10% less of the CYP2B6 substrate, wherein the CYP2B6 substrate is bupropion or efavirenz, or a pharmaceutically acceptable salt thereof. In some embodiments, provided is a method of administering 2 mg-8 mg of dasotraline, or a pharmaceutically acceptable salt thereof, once daily to a subject in need thereof, and also in need of a CYP2B6 substrate, comprising concomitantly administering 2 mg-8 mg of dasotraline, or a pharmaceutically acceptable salt thereof, once daily and at least 40% less of the CYP2B6 substrate, wherein the CYP2B6 substrate is bupropion or efavirenz, or a pharmaceutically acceptable salt thereof.

In some embodiments, provided is a method of treating a central nervous system (CNS) disorder, in a subject in need thereof, comprising concomitantly administering an increase from a therapeutically effective dose of dasotraline, or a pharmaceutically acceptable salt thereof, and a therapeutically effective amount of a CYP2B6 inducer. In some embodiments, provided is a method of treating a central nervous system (CNS) disorder, in a subject in need thereof, comprising concomitantly administering at least a 10% increase from a therapeutically effective dose of dasotraline, or a pharmaceutically acceptable salt thereof, and a therapeutically effective amount of a CYP2B6 inducer, wherein the CYP2B6 inducer is carbamazepine, efavirenz, rifampin, or ritonavir, or a pharmaceutically acceptable salt thereof. In some embodiments, provided is a method of treating a central nervous system (CNS) disorder, in a subject in need thereof, comprising concomitantly administering at least a 40% increase from a therapeutically effective dose of dasotraline, or a pharmaceutically acceptable salt thereof, and a therapeutically effective amount of a CYP2B6 inducer, wherein the CYP2B6 inducer is carbamazepine, efavirenz, rifampin, or ritonavir, or a pharmaceutically acceptable salt thereof.

In some embodiments, provided is a method of treating a central nervous system (CNS) disorder with a therapeutically effective dose of dasotraline, or a pharmaceutically acceptable salt thereof, in a subject in need thereof and also in need of a therapeutically effective amount of a CYP2B6 inducer, comprising increasing the dose or dose frequency of dasotraline during concomitant administration with the CYP2B6 inducer. In some embodiments, provided is a method of treating a central nervous system (CNS) disorder with a therapeutically effective dose of dasotraline, or a pharmaceutically acceptable salt thereof, in a subject in need thereof and also in need of a therapeutically effective amount of a CYP2B6 inducer, comprising increasing the dose frequency of dasotraline by at least 10% during concomitant administration with the CYP2B6 inducer, wherein the CYP2B6 inducer is carbamazepine, efavirenz, rifampin, or ritonavir, or a pharmaceutically acceptable salt thereof. In some embodiments, provided is a method of treating a central nervous system (CNS) disorder with a therapeutically effective dose of dasotraline, or a pharmaceutically acceptable salt thereof, in a subject in need thereof and also in need of a therapeutically effective amount of a CYP2B6 inducer, comprising increasing the dose of dasotraline by at least 10% during concomitant administration with the CYP2B6 inducer, wherein the CYP2B6 inducer is carbamazepine, efavirenz, rifampin, or ritonavir, or a pharmaceutically acceptable salt thereof. In some embodiments, provided is a method of treating a central nervous system (CNS) disorder with a therapeutically effective dose of dasotraline, or a pharmaceutically acceptable salt thereof, in a subject in need thereof and also in need of a therapeutically effective amount of a CYP2B6 inducer, comprising increasing the dose frequency of dasotraline by at least 40% during concomitant administration with the CYP2B6 inducer, wherein the CYP2B6 inducer is carbamazepine, efavirenz, rifampin, or ritonavir, or a pharmaceutically acceptable salt thereof. In some embodiments, provided is a method of treating a central nervous system (CNS) disorder with a therapeutically effective dose of dasotraline, or a pharmaceutically acceptable salt thereof, in a subject in need thereof and also in need of a therapeutically effective amount of a CYP2B6 inducer, comprising increasing the dose of dasotraline by at least 40% during concomitant administration with the CYP2B6 inducer, wherein the CYP2B6 inducer is carbamazepine, efavirenz, rifampin, or ritonavir, or a pharmaceutically acceptable salt thereof.

In some embodiments, provided is a method of treating a central nervous system (CNS) disorder, in a subject in need thereof, comprising administering once daily 2 mg-8 mg of dasotraline, or a pharmaceutically acceptable salt thereof, and avoiding a CYP2B6 inducer. In some embodiments, provided is a method of treating a central nervous system (CNS) disorder, in a subject in need thereof, comprising administering once daily 2 mg-8 mg of dasotraline, or a pharmaceutically acceptable salt thereof, and avoiding a CYP2B6 inducer, wherein the CYP2B6 inducer is carbamazepine, efavirenz, rifampin, or ritonavir, or a pharmaceutically acceptable salt thereof.

In some embodiments, provided is a method of treating a central nervous system (CNS) disorder with 2 mg-8 mg of dasotraline, or a pharmaceutically acceptable salt thereof, once daily in a subject in need thereof and also in need of a CYP2B6 inducer, comprising avoiding the CYP2B6 inducer during administration of dasotraline, or a pharmaceutically acceptable salt thereof. In some embodiments, provided is a method of treating a central nervous system (CNS) disorder with 2 mg-8 mg of dasotraline, or a pharmaceutically acceptable salt thereof, once daily in a subject in need thereof and also in need of a CYP2B6 inducer, comprising avoiding the CYP2B6 inducer during administration of dasotraline, or a pharmaceutically acceptable salt thereof, wherein the CYP2B6 inducer is carbamazepine, efavirenz, rifampin, or ritonavir, or a pharmaceutically acceptable salt thereof.

In some embodiments, provided is a method of treating a central nervous system (CNS) disorder, in a subject in need thereof, comprising administering once daily 2 mg-8 mg of dasotraline, or a pharmaceutically acceptable salt thereof, and discontinuing administration of a CYP2B6 inducer. In some embodiments, provided is a method of treating a central nervous system (CNS) disorder, in a subject in need thereof, comprising administering once daily 2 mg-8 mg of dasotraline, or a pharmaceutically acceptable salt thereof, and discontinuing administration of a CYP2B6 inducer, wherein the CYP2B6 inducer is carbamazepine, efavirenz, rifampin, or ritonavir, or a pharmaceutically acceptable salt thereof.

In some embodiments, provided is a method of treating a central nervous system (CNS) disorder with 2 mg-8 mg of dasotraline, or a pharmaceutically acceptable salt thereof, once daily in a subject in need thereof and also in need of a CYP2B6 inducer, comprising discontinuing administration of the CYP2B6 inducer during administration of dasotraline, or a pharmaceutically acceptable salt thereof. In some embodiments, provided is a method of treating a central nervous system (CNS) disorder with 2 mg-8 mg of dasotraline, or a pharmaceutically acceptable salt thereof, once daily in a subject in need thereof and also in need of a CYP2B6 inducer, comprising discontinuing administration of the CYP2B6 inducer during administration of dasotraline, or a pharmaceutically acceptable salt thereof, wherein the CYP2B6 inducer is carbamazepine, efavirenz, rifampin, or ritonavir, or a pharmaceutically acceptable salt thereof. In some embodiments, provided is a method of treating a central nervous system (CNS) disorder while reducing potential drug-drug interactions, in a subject in need thereof, comprising concomitantly administering an increase of dasotraline, or a pharmaceutically acceptable salt thereof, and an effective amount of a CYP2B6 inducer. In some embodiments, provided is a method of treating a central nervous system (CNS) disorder while reducing potential drug-drug interactions, in a subject in need thereof, comprising concomitantly administering at least a 10% increase of dasotraline, or a pharmaceutically acceptable salt thereof, and an effective amount of a CYP2B6 inducer, wherein the CYP2B6 inducer is carbamazepine, efavirenz, rifampin, or ritonavir, or a pharmaceutically acceptable salt thereof. In some embodiments, provided is a method of treating a central nervous system (CNS) disorder while reducing potential drug-drug interactions, in a subject in need thereof, comprising concomitantly administering at least a 40% increase of dasotraline, or a pharmaceutically acceptable salt thereof, and an effective amount of a CYP2B6 inducer, wherein the CYP2B6 inducer is carbamazepine, efavirenz, rifampin, or ritonavir, or a pharmaceutically acceptable salt thereof.

In some embodiments, provided is a method of administering dasotraline, or a pharmaceutically acceptable salt thereof, to a subject in need thereof, and also in need of a CYP2B6 inducer, comprising concomitantly administering the CYP2B6 inducer and more than a normally effective amount of dasotraline, or a pharmaceutically acceptable salt thereof. In some embodiments, provided is a method of administering dasotraline, or a pharmaceutically acceptable salt thereof, to a subject in need thereof, and also in need of a CYP2B6 inducer, comprising concomitantly administering the CYP2B6 inducer and at least 10% more of an effective amount of dasotraline, or a pharmaceutically acceptable salt thereof, wherein the CYP2B6 inducer is carbamazepine, efavirenz, rifampin, or ritonavir, or a pharmaceutically acceptable salt thereof. In some embodiments, provided is a method of administering dasotraline, or a pharmaceutically acceptable salt thereof, to a subject in need thereof, and also in need of a CYP2B6 inducer, comprising concomitantly administering the CYP2B6 inducer and at least 40% more of an effective amount of dasotraline, or a pharmaceutically acceptable salt thereof, wherein the CYP2B6 inducer is carbamazepine, efavirenz, rifampin, or ritonavir, or a pharmaceutically acceptable salt thereof.

In some embodiments, provided is a method of treating a central nervous system (CNS) disorder, in a subject in need thereof, comprising concomitantly administering once daily 2 mg-8 mg of dasotraline, or a pharmaceutically acceptable salt thereof, and a therapeutically effective amount of a CYP2B6 time-dependent inhibitor (TDI), wherein the CYP2B6 inhibitor is a medicament.

In some embodiments, provided is a method of treating a central nervous system (CNS) disorder, in a subject in need thereof, comprising concomitantly administering once daily 2 mg-8 mg of dasotraline, or a pharmaceutically acceptable salt thereof, and a therapeutically effective amount of a direct inhibitor of CYP2D6, CYP2C19 and CYP3A, wherein the direct inhibitor of CYP2D6, CYP2C19 and CYP3A is a medicament.

In some preferred embodiments, the therapeutically effective amount of dasotraline is 2 mg-8 mg once daily. In some preferred embodiments, the therapeutically effective amount of dasotraline is 2 mg, 4 mg, 6 mg, or 8 mg once daily.

In some embodiments, a 5% decrease from a normally effective amount of CYP2B6 substrate is concomitantly administered with dasotraline, or a pharmaceutically acceptable salt thereof. In some embodiments, a 10% decrease from a normally effective amount of CYP2B6 substrate is concomitantly administered with dasotraline, or a pharmaceutically acceptable salt thereof. In some embodiments, a 15% decrease from a normally effective amount of CYP2B6 substrate is concomitantly administered with dasotraline, or a pharmaceutically acceptable salt thereof. In some embodiments, a 20% decrease from a normally effective amount of CYP2B6 substrate is concomitantly administered with dasotraline, or a pharmaceutically acceptable salt thereof. In some embodiments, a 25% decrease from a normally effective amount of CYP2B6 substrate is concomitantly administered with dasotraline, or a pharmaceutically acceptable salt thereof. In some embodiments, a 30% decrease from a normally effective amount of CYP2B6 substrate is concomitantly administered with dasotraline, or a pharmaceutically acceptable salt thereof. In some embodiments, a 35% decrease from a normally effective amount of CYP2B6 substrate is concomitantly administered with dasotraline, or a pharmaceutically acceptable salt thereof. In some embodiments, a 40% decrease from a normally effective amount of CYP2B6 substrate is concomitantly administered with dasotraline, or a pharmaceutically acceptable salt thereof. In some embodiments, a 45% decrease from a normally effective amount of CYP2B6 substrate is concomitantly administered with dasotraline, or a pharmaceutically acceptable salt thereof. In some embodiments, a 50% decrease from a normally effective amount of CYP2B6 substrate is concomitantly administered with dasotraline, or a pharmaceutically acceptable salt thereof. In some embodiments, a 55% decrease from a normally effective amount of CYP2B6 substrate is concomitantly administered with dasotraline, or a pharmaceutically acceptable salt thereof. In some embodiments, a 60% decrease from a normally effective amount of CYP2B6 substrate is concomitantly administered with dasotraline, or a pharmaceutically acceptable salt thereof. In some embodiments, a 65% decrease from a normally effective amount of CYP2B6 substrate is concomitantly administered with dasotraline, or a pharmaceutically acceptable salt thereof. In some embodiments, a 70% decrease from a normally effective amount of CYP2B6 substrate is concomitantly administered with dasotraline, or a pharmaceutically acceptable salt thereof. In some embodiments, a 75% decrease from a normally effective amount of CYP2B6 substrate is concomitantly administered with dasotraline, or a pharmaceutically acceptable salt thereof.

In some embodiments, the dose of a CYP2B6 substrate is decreased by at least 5% during concomitant administration with dasotraline, or a pharmaceutically acceptable salt thereof. In some embodiments, the dose of a CYP2B6 substrate is decreased by at least 10% during concomitant administration with dasotraline, or a pharmaceutically acceptable salt thereof. In some embodiments, the dose of a CYP2B6 substrate is decreased by at least 15% during concomitant administration with dasotraline, or a pharmaceutically acceptable salt thereof. In some embodiments, the dose of a CYP2B6 substrate is decreased by at least 20% during concomitant administration with dasotraline, or a pharmaceutically acceptable salt thereof. In some embodiments, the dose of a CYP2B6 substrate is decreased by at least 25% during concomitant administration with dasotraline, or a pharmaceutically acceptable salt thereof. In some embodiments, the dose of a CYP2B6 substrate is decreased by at least 30% during concomitant administration with dasotraline, or a pharmaceutically acceptable salt thereof. In some embodiments, the dose of a CYP2B6 substrate is decreased by at least 35% during concomitant administration with dasotraline, or a pharmaceutically acceptable salt thereof. In some embodiments, the dose of a CYP2B6 substrate is decreased by at least 40% during concomitant administration with dasotraline, or a pharmaceutically acceptable salt thereof. In some embodiments, the dose of a CYP2B6 substrate is decreased by at least 45% during concomitant administration with dasotraline, or a pharmaceutically acceptable salt thereof. In some embodiments, the dose of a CYP2B6 substrate is decreased by at least 50% during concomitant administration with dasotraline, or a pharmaceutically acceptable salt thereof. In some embodiments, the dose of a CYP2B6 substrate is decreased by at least 55% during concomitant administration with dasotraline, or a pharmaceutically acceptable salt thereof. In some embodiments, the dose of a CYP2B6 substrate is decreased by at least 60% during concomitant administration with dasotraline, or a pharmaceutically acceptable salt thereof. In some embodiments, the dose of a CYP2B6 substrate is decreased by at least 65% during concomitant administration with dasotraline, or a pharmaceutically acceptable salt thereof. In some embodiments, the dose of a CYP2B6 substrate is decreased by at least 70% during concomitant administration with dasotraline, or a pharmaceutically acceptable salt thereof. In some embodiments, the dose of a CYP2B6 substrate is decreased by at least 75% during concomitant administration with dasotraline, or a pharmaceutically acceptable salt thereof.

In some embodiments, the CYP2B6 substrate is amitriptyline, antipyrine, artemether, artemisinin, asunaprevir, banoxantrone, benzphetamine, brivaracetam, brompheniramine, bupropion, cinnarizine, cisapride, clobazam, clomethiazole, clomipramine, clopidogrel, clotiazepam, cyclophosphamide, desipramine, dextromethorphan, diazepam, diclofenac, domperidone, dosulepin, doxepin, efavirenz, enasidenib, epinastine, erythromycin, estrone, ethylmorphine, flunitrazepam, fluoxetine, halothane, ifosfamide, imipramine, irinotecan, isoflurane, ixazomib, ketamine, ketobemidone, lidocaine, loperamide, lorcaserin, malathion, melitracen, meperidine, mephenytoin, methadone, methoxyflurane, methylphenobarbital, methyltestosterone, mexiletine, mianserin, midazolam, nevirapine, nicotine, nortriptyline, ospemifene, perampanel, perhexiline, permethrin, perphenazine, prasugrel, promethazine, propofol, ritonavir, romidepsin, ropivacaine, selegiline, seratrodast, sertraline, sevoflurane, tamoxifen, temazepam, testosterone, testosterone cypionate, testosterone enanthate, testosterone undecanoate, tramadol, tretinoin, trimipramine, valproic acid, velpatasvir, verapamil, or, vortioxetine, or a pharmaceutically acceptable salt thereof. In some preferred embodiments, the CYP2B6 substrate is bupropion or efavirenz, or a pharmaceutically acceptable salt thereof.

In some embodiments, a 5% increase from a normally effective amount of dasotraline, or a pharmaceutically acceptable salt thereof, is concomitantly administered with a CYP2B6 inducer. In some embodiments, a 10% increase from a normally effective amount of dasotraline, or a pharmaceutically acceptable salt thereof, is concomitantly administered with a CYP2B6 inducer. In some embodiments, a 15% increase from a normally effective amount of dasotraline, or a pharmaceutically acceptable salt thereof, is concomitantly administered with a CYP2B6 inducer. In some embodiments, a 20% increase from a normally effective amount of dasotraline, or a pharmaceutically acceptable salt thereof, is concomitantly administered with a CYP2B6 inducer. In some embodiments, a 25% increase from a normally effective amount of dasotraline, or a pharmaceutically acceptable salt thereof, is concomitantly administered with a CYP2B6 inducer. In some embodiments, a 30% increase from a normally effective amount of dasotraline, or a pharmaceutically acceptable salt thereof, is concomitantly administered with a CYP2B6 inducer. In some embodiments, a 35% increase from a normally effective amount of dasotraline, or a pharmaceutically acceptable salt thereof, is concomitantly administered with a CYP2B6 inducer. In some embodiments, a 40% increase from a normally effective amount of dasotraline, or a pharmaceutically acceptable salt thereof, is concomitantly administered with a CYP2B6 inducer. In some embodiments, a 45% increase from a normally effective amount of dasotraline, or a pharmaceutically acceptable salt thereof, is concomitantly administered with a CYP2B6 inducer. In some embodiments, a 50% increase from a normally effective amount of dasotraline, or a pharmaceutically acceptable salt thereof, is concomitantly administered with a CYP2B6 inducer. In some embodiments, a 55% increase from a normally effective amount of dasotraline, or a pharmaceutically acceptable salt thereof, is concomitantly administered with a CYP2B6 inducer. In some embodiments, a 60% increase from a normally effective amount of dasotraline, or a pharmaceutically acceptable salt thereof, is concomitantly administered with a CYP2B6 inducer. In some embodiments, a 65% increase from a normally effective amount of dasotraline, or a pharmaceutically acceptable salt thereof, is concomitantly administered with a CYP2B6 inducer. In some embodiments, a 70% increase from a normally effective amount of dasotraline, or a pharmaceutically acceptable salt thereof, is concomitantly administered with a CYP2B6 inducer. In some embodiments, a 75% increase from a normally effective amount of dasotraline, or a pharmaceutically acceptable salt thereof, is concomitantly administered with a CYP2B6 inducer.

In some embodiments, the dose of dasotraline, or a pharmaceutically acceptable salt thereof, is increased by at least 5% during concomitant administration with a CYP2B6 inducer. In some embodiments, the dose of dasotraline, or a pharmaceutically acceptable salt thereof, is increased by at least 10% during concomitant administration with a CYP2B6 inducer. In some embodiments, the dose of dasotraline, or a pharmaceutically acceptable salt thereof, is increased by at least 15% during concomitant administration with a CYP2B6 inducer. In some embodiments, the dose of dasotraline, or a pharmaceutically acceptable salt thereof, is increased by at least 20% during concomitant administration with a CYP2B6 inducer. In some embodiments, the dose of dasotraline, or a pharmaceutically acceptable salt thereof, is increased by at least 25% during concomitant administration with a CYP2B6 inducer. In some embodiments, the dose of dasotraline, or a pharmaceutically acceptable salt thereof, is increased by at least 30% during concomitant administration with a CYP2B6 inducer. In some embodiments, the dose of dasotraline, or a pharmaceutically acceptable salt thereof, is increased by at least 35% during concomitant administration with a CYP2B6 inducer. In some embodiments, the dose of dasotraline, or a pharmaceutically acceptable salt thereof, is increased by at least 40% during concomitant administration with a CYP2B6 inducer. In some embodiments, the dose of dasotraline, or a pharmaceutically acceptable salt thereof, is increased by at least 45% during concomitant administration with a CYP2B6 inducer. In some embodiments, the dose of dasotraline, or a pharmaceutically acceptable salt thereof, is increased by at least 50% during concomitant administration with a CYP2B6 inducer. In some embodiments, the dose of dasotraline, or a pharmaceutically acceptable salt thereof, is increased by at least 55% during concomitant administration with a CYP2B6 inducer. In some embodiments, the dose of dasotraline, or a pharmaceutically acceptable salt thereof, is increased by at least 60% during concomitant administration with a CYP2B6 inducer. In some embodiments, the dose of dasotraline, or a pharmaceutically acceptable salt thereof, is increased by at least 65% during concomitant administration with a CYP2B6 inducer. In some embodiments, the dose of dasotraline, or a pharmaceutically acceptable salt thereof, is increased by at least 70% during concomitant administration with a CYP2B6 inducer. In some embodiments, the dose of dasotraline, or a pharmaceutically acceptable salt thereof, is increased by at least 75% during concomitant administration with a CYP2B6 inducer.

In some embodiments, the CYP2B6 inducer is armodafinil, artemether, atorvastatin, carbamazepine, cerivastatin, clofibrate, clotrimazole, cyclophosphamide, dabrafenib, dexamethasone, efavirenz, enasidenib, fluvastatin, fosphenytoin, idelalisib, isavuconazole, isoflurane, ivosidenib, letermovir, lumacaftor, metamizole, midostaurin, modafinil, nevirapine, nicardipine, nifedipine, nilotinib, olaparib, permethrin, phenobarbital, phenytoin, pitolisant, primidone, rifabutin, rifampicin, rifamycin, rifapentine, ritonavir, simvastatin, sulfinpyrazone, testosterone, testosterone cypionate, testosterone enanthate, testosterone undecanoate, troglitazone, vemurafenib, verapamil, or a pharmaceutically acceptable salt thereof. In a preferred embodiment, the CYP2B6 inducer is carbamazepine, efavirenz, rifampin, or ritonavir, or a pharmaceutically acceptable salt thereof.

In some embodiments, the central nervous system disorder is a disorder identified in the Diagnostic and Statistical Manual of Mental Disorders, 5thEd., American Psychiatric Association (2013) (“DSM-5”). In some embodiments, the central nervous system disorder is a neurodegenerative disease (e.g., Alzheimer's disease, Parkinson's disease, amyotrophic lateral sclerosis, etc.), a neuropsychiatric disease (e.g., schizophrenia, anxieties, such as general anxiety disorder, etc.), and or a disorder (e.g., depression, bipolar disorder, manic conditions, attention disorders, feeding or eating disorders, etc.). In some preferred embodiments, the central nervous system disorder is an attention disorder or a feeding or eating disorder. In some preferred embodiments, the central nervous system disorder is an attention disorder. In some preferred embodiments, the central nervous system disorder is a feeding or eating disorder.

In some preferred embodiments, the central nervous system disorder is attention deficit disorder (ADD), attention deficit disorder with hyperactivity (ADDH), attention deficit hyperactivity disorder (ADHD), or equivalent disorders identified in the Diagnostic and Statistical Manual of Mental Disorders, 5thEd., American Psychiatric Association (2013) (“DSM-5”). In some preferred embodiments, the central nervous system disorder is attention deficit disorder (ADD), attention deficit disorder with hyperactivity (ADDH), or attention deficit hyperactivity disorder (ADHD).

In some preferred embodiments, the central nervous system disorder is pica, rumination disorder, avoidant/restrictive food intake disorder, anorexia nervosa, bulimia nervosa, binge-eating disorder, other specified feeding or eating disorder, or unspecified feeding or eating disorder. In some preferred embodiments, the central nervous system disorder is binge-eating disorder.

### EXAMPLES

The inhibitory potency of dasotraline was determined in vitro by measuring the activity of CYP2B6 in human liver microsomes in the presence and absence of dasotraline. In vitro studies with human liver microsomes indicated that dasotraline is a direct competitive inhibitor of cytochrome P450 (CYP) 2B6 with an inhibition constant value of 0.22 μM. Furthermore, dasotraline was determined to be a time-dependent inhibitor of CYP2B6 with an apparent inactivation constant of 0.12 μM and a maximal inactivation rate constant of 0.15 min-1. Based on the Food and Drug Administration Draft Guidance: Drug Interaction Studies (February 2012) and the prediction using physiologically-based PK modeling and simulation there is a potential for dasotraline to inhibit the metabolism pathway of CYP2B6 substrates through time-dependent inhibition. Therefore, the effect of dasotraline on the PK of a CYP2B6 substrate (ie, efavirenz) was studied clinically.

### Example 1. A Randomized, 2-Way Crossover, Drug Interaction Study to Evaluate the Effects of Dasotraline on the Single-Dose Pharmacokinetics of Efavirenz in Healthy Subjects

A randomized, 2-way crossover study to evaluate the effect of dasotraline on the pharmacokinetics (PK) of efavirenz, a sensitive cytochrome P450 2B6 (CYP2B6) substrate, and to assess the safety and tolerability of a single dose of dasotraline given concurrently with efavirenz was conducted.

Methodology: The study enrolled 29 healthy, non-smoking male and female subjects. All subjects were randomized to one of two treatment sequences: (A) Single doses of placebo and 400 mg efavirenz followed 28 days later by single doses of 24 mg dasotraline and 400 mg efavirenz; or (B) Single doses of 24 mg dasotraline and 400 mg efavirenz followed 28 days later by single doses of placebo and 400 mg efavirenz. Dasotraline and placebo were administered 6 hours prior to the planned dosing time of efavirenz. A screening period of approximately 4 weeks was followed by 2 treatment periods, each of which included approximately 78 hours in-clinic confinement followed by 3 outpatient follow-up visits. Dose administration occurred on Day 1 and Day 29. During the first treatment period, subjects were admitted to the clinic on Day −1, the day before first study drug administration (Day 1), and remained in clinic until discharge on Day 3. Subjects returned to the clinic for 3 subsequent visits (Days 5, 7, and 14) for PK sample collection. Following a washout period (consisting of 28 non-dosing days), subjects returned and were readmitted to the clinic for the second treatment period on Day 28, the day before the second study drug administration (Day 29), and remained in clinic until discharge on Day 31. Subjects returned to the clinic for 3 subsequent visits (Days 33, 35, and 42) for PK sample collection. Day 42 was the End of Study visit.

Evaluation: Blood for serial PK sampling was obtained from all subjects prior to efavirenz administration (pre-dose) and at specified time points after efavirenz administration on Days 1 and 29 for each treatment sequence. The recorded times for dasotraline PK plasma sample collections were relative to efavirenz dosing time. The primary parameters for efavirenz analysis were maximum observed plasma concentration (Cₘₐₓ), time of maximum observed plasma concentration (tₘₐₓ), area under the concentration-time curve (AUC) from time 0 to time t (AUC₀₋ₜ), AUC from time 0 to the time of last measurable concentration (AUC₀₋ₗₐₛₜ), AUC from time 0 extrapolated to infinity (AUC_{0-inf}), terminal elimination rate constant (λ_{Z}), apparent terminal elimination half-life (t_{1/2}), apparent total clearance (CL/F), and apparent volume of distribution during the terminal elimination phase (V_{z}/F). The primary parameters for dasotraline analysis were Cₘₐₓ, AUC₀₋ₗₐₛₜ, AUC₀₋ₜ, and AUC_{0-inf}.

Statistical Methods: All analyses involving PK data (e.g., plasma concentrations, derived PK parameters, assessment of drug-drug interaction) were performed on the PK population. For plasma concentrations of dasotraline and efavirenz, descriptive statistics (e.g., number, arithmetic mean, arithmetic standard deviation, minimum, median, maximum, 25th percentile, 75th percentile, geometric mean, and geometric coefficient of variation) were listed and summarized by time point by treatment for the PK population. Mean plasma concentration-time profiles were plotted by treatment on linear and semi-logarithmic scales. Individual subject linear and semi-logarithmic concentration-time plots, with respective treatments presented together, were included in the appendices.

To evaluate the effect of dasotraline on the PK of efavirenz, a mixed effect analysis of variance (ANOVA) was applied for the primary endpoint on natural log-transformed Cₘₐₓ, AUC₀₋ₗₐₛₜ, AUC₀₋ₜ, and AUC_{0-inf} of efavirenz. The model included treatment, sequence, and period as fixed effects and subject nested within sequence as a random effect. Only subjects who completed both treatment periods were included in the overall ANOVA to allow for an intra-subject comparison to be conducted. Geometric least squares (LS) means were estimated for each parameter of each treatment. For each parameter, the ratio of geometric LS means for 24 mg dasotraline coadministered with 400 mg efavirenz (substrate+interacting drug as test treatment)

Results (summarized in Table 1 and Table 2): Mean efavirenz plasma concentrations at each time point were similar through the first 12 hours post-dose after concomitant dasotraline administration as compared to administration with placebo. Mean efavirenz plasma concentrations subsequently increased at time points 16 to 312 hours post-dose after concomitant dasotraline administration as compared to administration with placebo. Mean efavirenz Cₘₐₓ was similar after concomitant dasotraline administration (1510 ng/mL) as compared to administration with placebo (1560 ng/mL). Median tₘₐₓ was similar when efavirenz was administered concomitantly with dasotraline (4 hours, range 1 to 12 hours) compared to administration with placebo (approximately 3 hours, range 1 to 10 hours). Mean efavirenz AUC from time 0 to 24 hours post-dose (AUC₀₋₂₄) was similar after concomitant administration with dasotraline (19,000 h*ng/mL) compared to administration with placebo (17,700 h*ng/mL); however, mean overall exposures were increased after concomitant administration with dasotraline (AUC₀₋ₗₐₛₜ 112,000 h*ng/mL and AUC_{0-inf} 135,000 h*ng/mL) compared to administration with placebo (AUC₀₋ₗₐₛₜ 78,200 h*ng/mL and AUC_{0-inf} 87,900 h*ng/mL). Mean terminal half-life was 143 hours when efavirenz was administered with placebo. Mean terminal half-life increased to 161 hours when administered concomitantly with dasotraline. Mean efavirenz CL/F was 4.60 L/h when efavirenz was administered with placebo. Mean CL/F decreased to 3.08 L/h when administered concomitantly with dasotraline. Mean V_{z}/F was 759 L when efavirenz was administered with placebo. Mean efavirenz V_{z}/F decreased to 525 L when administered concomitantly with dasotraline.

The geometric LS mean ratios for Cₘₐₓ, AUC₀₋₂₄, AUC₀₋ₗₐₛₜ, and AUC_{0-inf} values for efavirenz administered concomitantly with dasotraline as compared to administration with placebo were 98.5%, 107.5%, 143.1%, and 153.5%, respectively. The overall exposure (AUCs) 90% CI range did not include 1; this increase in exposure was deemed statistically significant. Mean (SD) plasma PK parameters for dasotraline are presented in Table 2.

TABLE 1
Summary of Plasma Pharmacokinetic (PK) Parameters for Efavirenz after
Administration of Efavirenz Concomitantly with Dasotraline or Placebo

| | Arithmetic Mean (SD) | | |
|---|---|---|---|
| | | Efavirenz + Dasotraline | Efavirenz + Placebo |
| PK Parameter* | | n = 27 | n = 28 |

| | | | | |
|---|---|---|---|---|
| AUC₀₋ₗₐₛₜ (h*ng/mL) | 113,000 | (28,200) | 80,400 | (19,200) |
| AUC₀₋₂₄ (h*ng/mL) | 19,500 | (5,300) | 18,200 | (4,450) |

| | | |
|---|---|---|
| AUC_{0-inf} (h*ng/mL) | 138,000 (35,300) (n = 19) | 92,000 (22,100) (n = 22) |

| | | | | |
|---|---|---|---|---|
| Cₘₐₓ (ng/mL) | 1,510 | (529) | 1,560 | (520) |
| tₘₐₓ (h)-median range | 4.00 | (1.00-12.0) | 3.05 | (1.00-10.0) |

| | | | |
|---|---|---|---|
| t_{1/2} (h) | 161 (84.6) (n = 26) | 143 | (61.5) |

| | | |
|---|---|---|
| CL/F (L/h) | 3.08 (0.753) (n = 19) | 4.60 (1.10) (n = 22) |
| V_{z}/F (L) | 525 (190) (n = 19) | 759 (251) (n = 22) |

TABLE 2
Summary of Plasma Pharmacokinetic Parameters for Dasotraline

| | Arithmetic Mean (SD) | |
|---|---|---|
| | Efavirenz + Dasotraline | |
| PK Parameter* | n = 27 | |
| AUC₀₋ₗₐₛₜ (h*ng/mL) | 1170 | (301) |
| AUC₀₋₂₄ (h*ng/mL) | 138 | (27.6) |
| AUC_{0-inf} (h*ng/mL) | 1270 (428) (n = 26) | |
| Cₘₐₓ (ng/mL) | 8.92 | (1.73) |

Abbreviations: AUC₀₋₂₄: area under the plasma concentration-time curve from time zero to 24 hours post-dose; AUC_{0-inf}: area under the plasma concentration-time curve from time 0 extrapolated to infinity; AUG)-last area under the plasma concentration-time curve from time 0 to the time of last measurable concentration; CL/F: apparent total clearance; Cₘₐₓ: maximum observed plasma concentration; n: number of subjects; t_{1/2}: apparent terminal elimination half-life; tₘₐₓ: time of maximum observed plasma concentration; SD: standard deviation; Vz/F: apparent volume of distribution during the terminal elimination phase.

Conclusion: The results of this study support the potential for dasotraline to inhibit the metabolism pathway of CYP2B6 substrates (e.g., efavirenz) through time-dependent inhibition. Efavirenz Cₘₐₓ, tₘₐₓ, and mean plasma concentrations for serial timepoints through 12 hours post-dose were similar when administered concomitantly with dasotraline or with placebo. However, evidence of dasotraline inhibiting the metabolism pathway of efavirenz was observed based on an increase in mean efavirenz plasma concentrations for serial time points from 16 to 312 hours post-dose. After concomitant administration of efavirenz with dasotraline, an increase in overall exposure (AUC₀₋₂₄, AUC₀₋ₗₐₛₜ, and AUC_{0-inf}), an increase in mean terminal half-life, and decreases in CL/F and V_{z}/F were observed as compared to efavirenz and placebo.

A Randomized, 2-Way Crossover, Drug Interaction Study to Evaluate the Effects of Dasotraline on the Single-Dose Pharmacokinetics of Efavirenz in Healthy Subjects: Statistical analysis of PK parameters for efavirenz administered concomitantly with dasotraline (test) and efavirenz administered with placebo (reference) showed no statistical difference in Cₘₐₓ, as the 90% CIs of the ratios of geometric LS means were contained within the interval 0.80 to 1.25. Exposure for the first 24 hours post-dose (AUC₀₋₂₄) showed a 7.5% increase in efavirenz plasma concentration in the presence of dasotraline versus placebo, with a ratio of geometric LS means (%) of 107.5 (90% CI: 103.0, 112.2). Analysis of AUC₀₋ₗₐₛₜ and AUC_{0-inf} showed approximately 43% and 54% increase in the presence of dasotraline, respectively, with ratios of geometric LS means (%) of 143.1 (90% CI: 135.7, 150.9) and 153.5 (90% CI: 144.0, 163.7), respectively. The increases in exposure (AUCs) were deemed statistically significant.

1.-18. (canceled)

19. A method of treating a central nervous system (CNS) disorder, in a subject in need thereof, comprising concomitantly administering once daily 2 mg-8 mg of dasotraline, or a pharmaceutically acceptable salt thereof, and a therapeutically effective amount of a CYP2B6 inhibitor, wherein the CYP2B6 inhibitor is a medicament.

20.-25. (canceled)

26. A method of administering 2 mg-8 mg of dasotraline, or a pharmaceutically acceptable salt thereof, once daily to a subject in need thereof, and also in need of a CYP2B6 substrate, comprising concomitantly administering once daily 2 mg-8 mg of dasotraline, or a pharmaceutically acceptable salt thereof, and 10% less of the CYP2B6 substrate, wherein the CYP2B6 substrate is bupropion or efavirenz, or a pharmaceutically acceptable salt thereof.

27. A method of administering dasotraline, or a pharmaceutically acceptable salt thereof, to a subject in need thereof, and also in need of a CYP2B6 inducer, comprising concomitantly administering the CYP2B6 inducer and 10% more of an effective amount of dasotraline, or a pharmaceutically acceptable salt thereof, wherein the CYP2B6 inducer is carbamazepine, efavirenz, rifampin, or ritonavir, or a pharmaceutically acceptable salt thereof.

28. The method according to claim 26 wherein said administering 2 mg-8 mg of dasotraline treats a central nervous system (CNS) disorder.

29. The method of claim 28, wherein the central nervous system (CNS) disorder is attention deficit disorder (ADD), attention deficit disorder with hyperactivity (ADDH), or attention deficit hyperactivity disorder (ADHD).

30. The method according to claim 26 wherein said administering 2 mg-8 mg of dasotraline reduces a risk of seizure.

31. The method according to claim 27 wherein said administering 2 mg-8 mg of dasotraline treats a central nervous system (CNS) disorder.

32. The method of claim 31, wherein the central nervous system (CNS) disorder is attention deficit disorder (ADD), attention deficit disorder with hyperactivity (ADDH), or attention deficit hyperactivity disorder (ADHD).

33. The method according to claim 19 wherein said medicament is a direct inhibitor of CYP2D6, CYP2C19 and CYP3A.

34. The method of claim 33, wherein the central nervous system (CNS) disorder is attention deficit disorder (ADD), attention deficit disorder with hyperactivity (ADDH), or attention deficit hyperactivity disorder (ADHD).
